(19) United States
(12) Patent Application Publication
(10) Pub. No.: US 2020/0281523 A1
(43) Pub. Date: Sep. 10, 2020

(54) APPARATUS FOR MONITORING A SUBJECT

(71) Applicant: EarlySense Ltd., Ramat Gan (IL)

(72) Inventors: Veronica MAIDEL, Givatayim (IL); Dalia ARGAMAN, Hod-Hasharon (IL); Zvika SHINAR, Binyamina (IL); Avner HALPERIN, Ramat Gan (IL)

(21) Appl. No.: 16/812,577

(22) Filed: Mar. 9, 2020

Related U.S. Application Data
(60) Provisional application No. 62/816,127, filed on Mar. 10, 2019.

(51) Int. Cl. A61B 5/00; A61B 5/0205

(52) U.S. Cl. CPC A61B 5/4842; A61B 5/0205; A61B 5/7278; A61B 5/024; A61B 5/4812; A61B 5/6892; A61B 5/746; A61B 5/4809

(57) ABSTRACT

A sensor monitors a subject and generates a signal. A computer processor receives the signal, derives therefrom data related to a heart rate of the subject and/or a respiration rate of the subject, derives from the signal at least one subject status indication that the subject is: awake and not moving, moving and asleep, not moving, asleep, awake and moving and in a bed, in a particular sleep stage while asleep, and/or has been continuously in a bed over a previous in-bed period of at least 15 hours, determines a threshold of at least 0.7 times an aerobic heart rate threshold and/or at least 0.7 times an aerobic respiratory rate threshold, and generates an alert if the signal indicates that the physiological parameter of the subject exceeds the threshold for an amount of time determined in response to the subject status indication. Other applications are also described.

### CROSS REFERENCE TO RELATED APPLICATIONS

The present application claims the priority of U.S. 62/816,127 to Maidel et al., filed Mar. 10, 2019, entitled, “Apparatus for monitoring a subject.”

The present application is related to an International Patent Application filed on even date herewith, entitled, “Apparatus for monitoring a subject.”

The above applications are incorporated herein by reference.

### FIELD OF THE INVENTION

The present invention relates generally to monitoring subjects, and specifically to predicting and monitoring abnormal physiological conditions by measurement, e.g., non-contact measurement, and analysis of characteristics of physiological and/or physical parameters.

### BACKGROUND

Many chronic diseases cause systemic changes in vital signs, such as breathing and heartbeat patterns, through a variety of physiological mechanisms. For example, common respiratory disorders, such as asthma, chronic obstructive pulmonary disease (COPD), sleep apnea and cystic fibrosis (CF), are direct modifiers of breathing and/or heartbeat patterns. Other chronic diseases, such as diabetes, epilepsy, and certain heart conditions (e.g., congestive heart failure (CHF)), are also known to modify cardiac and breathing activity. In the case of certain heart conditions, such modifications typically occur because of pathophysiologies related to fluid retention and general cardiovascular insufficiency. Other signs such as coughing and sleep restlessness are also known to be of importance in some clinical situations.

Many chronic diseases induce systemic effects on vital signs. For example, some chronic diseases interfere with normal breathing and cardiac processes during wakefulness and sleep, causing abnormal breathing and heartbeat patterns.

Breathing and heartbeat patterns may be modified via various direct and indirect physiological mechanisms, resulting in abnormal patterns related to the cause of modification. Some respiratory diseases, such as asthma, and some heart conditions, such as CHF, are direct breathing modifiers. Other metabolic abnormalities, such as hypoglycemia and other neurological pathologies affecting autonomic nervous system activity, are indirect breathing modifiers.

### SUMMARY OF THE INVENTION

For some applications of the present invention, a subject's respiration rate and/or heart rate is continuously monitored for a duration of time. Measuring heart rate and respiratory rate continuously allows for measuring a cumulative parameter of how long a subject's heart rate or respiratory rate is above or below a certain respective heart rate threshold or respiratory rate threshold during a given amount of time, which may be indicative of the onset or worsening of an adverse clinical condition. Typically, an alert is generated if the subject's heart rate and/or respiratory rate exceeds the threshold for a given cumulative amount of time out of a given time period.

There are circumstances in which a person's heart rate and/or respiratory rate may reach thresholds such as aerobic and anaerobic thresholds for reasons unrelated to adverse clinical conditions, e.g., while a person is exercising. However, during times when a subject is typically not active, e.g., when in a bed or on a resting surface, the subject's heart rate and/or respiratory rate being above certain thresholds, e.g., at least 0.7 times an aerobic heart rate threshold, or at least 0.7 times an aerobic respiratory rate threshold may be indicative of the onset or worsening of an adverse clinical condition. Using additional parameters such as sleep stage of a sleeping subject and motion information allows providing a timely, accurate alert with few false alarms.

There is therefore provided, in accordance with some applications of the present invention, apparatus for monitoring a subject, the apparatus including:

(A) a sensor configured to continuously monitor the subject, and to generate a sensor signal in response to the monitoring; and

(B) a computer processor, configured:
to receive the sensor signal,
to derive from the sensor signal data related to one or more physiological parameters of the subject selected from the group consisting of: a heart rate of the subject, and a respiration rate of the subject,
to determine a threshold selected from the group consisting of: at least 0.7 times an aerobic heart rate threshold, and at least 0.7 times an aerobic respiratory rate threshold, and
to generate an alert if the sensor signal indicates that the physiological parameter of the subject exceeds the threshold for a given percentage of time during a time period when the subject is not moving for at least 15 minutes.

For some applications, the sensor is configured to be disposed at a location selected from the group consisting of: upon a bed of the subject, and within the subject's bed.

For some applications, the sensor is configured to continuously monitor the subject, without contacting or viewing the subject, and to generate the sensor signal in response to the monitoring.

For some applications, the computer processor is configured to receive an age of the subject and, based on the age, to determine the threshold.

For some applications, the time period is at least 30 minutes.

For some applications, the threshold is selected from the group consisting of: at least 0.8 times an aerobic heart rate threshold, and at least 0.8 times an aerobic respiratory rate threshold.

For some applications, the threshold is selected from the group consisting of: at least an aerobic heart rate threshold and at least an aerobic respiratory rate threshold.

For some applications, the threshold is selected from the group consisting of: at least 0.7 times an anaerobic heart rate threshold, and at least 0.7 times an anaerobic respiratory rate threshold.

For some applications, the threshold is selected from the group consisting of: at least 0.8 times an anaerobic heart rate threshold, and at least 0.8 times an anaerobic respiratory rate threshold.

For some applications, the threshold is selected from the group consisting of: at least an anaerobic heart rate threshold, and at least an anaerobic respiratory rate threshold

There is further provided, in accordance with some applications of the present invention, apparatus for monitoring a subject, the apparatus including:

(A) a sensor configured to continuously monitor the subject, and to generate a sensor signal in response to the monitoring; and

(B) a computer processor, configured:
to receive the sensor signal,
to derive from the sensor signal data related to a physiological parameter of the subject selected from the group consisting of: a heart rate of the subject, and a respiratory rate of the subject,
determine a threshold selected from the group consisting of: (a) a resting heart rate threshold that is within a resting zone that is a resting heart rate zone of the subject, and (b) a resting respiratory rate threshold that is within a resting zone that is a resting respiratory rate zone of the subject, and
to generate an alert if the sensor signal indicates that the physiological parameter of the subject, during a portion of a time period, (a) is in the resting zone, and (b) exceeds the threshold, wherein the time period is at least 3 hours, and the portion of the time period is a percentage that is at least 70% of the time period.

For some applications, the sensor is configured to be disposed at a location selected from the group consisting of: upon a bed of the subject, and within the subject's bed.

For some applications, the computer processor is further configured to generate the alert if the sensor signal indicates that the physiological parameter of the subject, during the portion of the time period (a) is outside of the resting zone, and (b) exceeds the threshold.

For some applications, the computer processor is configured to receive an age of the subject and, based on the age, to determine the threshold.

For some applications:

a lower end of the resting heart rate zone is a resting heart rate of the subject that is 45-80 beats per minute,

the subject has a maximal heart rate, and

a higher end of the resting heart rate zone is above the lower end of the resting heart rate zone by up to 50% of the difference between the maximal heart rate of the subject and the resting heart rate of the subject.

For some applications, the selected threshold is the resting heart rate threshold, and the computer processor is configured to:
derive from the sensor signal data indicative of a heart rate of the subject while the subject is undergoing deep sleep; and
determine the resting heart rate threshold in response to the data indicative of the heart rate of the subject while the subject is undergoing deep sleep.

For some applications, the selected threshold is the resting respiratory rate threshold, and the computer processor is configured to:
derive from the sensor signal data indicative of a respiratory rate of the subject while the subject is undergoing deep sleep; and
determine the resting respiratory rate threshold in response to the data indicative of the respiratory rate of the subject while the subject is undergoing deep sleep.

For some applications, the selected threshold is the resting heart rate threshold, and the computer processor is configured to:
derive from the sensor signal data indicative of a lowest heart rate of the subject in a previous given time period; and
determine the resting heart rate threshold in response to the data indicative of the lowest heart rate of the subject.

For some applications, the given time period is 5-8 hours.

For some applications, the given time period is defined as extending from a first time of day to a second time of day.

For some applications, the selected threshold is the resting respiratory rate threshold, and the computer processor is configured to:
derive from the sensor signal data indicative of a lowest respiratory rate of the subject in a previous given time period; and
determine the resting respiratory rate threshold in response to the data indicative of the lowest respiratory rate of the subject.

For some applications, the given time period is 5-8 hours.

For some applications, the given time period extends from a first time of day to a second time of day.

There is further provided, in accordance with some applications of the present invention, apparatus for monitoring a subject, the apparatus including:

(A) a sensor, configured to continuously monitor the subject, and to generate a sensor signal in response to the monitoring; and

(B) a computer processor, configured:
to receive the sensor signal,
to derive from the sensor signal data related to one or more physiological parameters of the subject selected from the group consisting of: a heart rate of the subject, and a respiration rate of the subject,
to derive from the sensor signal at least one subject status indication selected from the group consisting of: an indication that the subject is moving and asleep, an indication that the subject is not moving, an indication that the subject is awake and not moving, an indication that the subject is asleep, an indication that the subject is awake and moving and in a bed, an indication of a particular sleep stage of the subject while the subject is asleep, and an indication that the subject has been continuously in a bed over a previous in-bed period of at least 15 hours,
to determine a threshold selected from the group consisting of: at least 0.7 times an aerobic heart rate threshold, and at least 0.7 times an aerobic respiratory rate threshold, and
to generate an alert if the sensor signal indicates that the physiological parameter of the subject exceeds the threshold for an amount of time determined in response to the subject status indication.

For some applications, the sensor is configured to be disposed at a location selected from the group consisting of: upon the subject's bed, and within the subject's bed.

For some applications, the computer processor is configured to receive an age of the subject and, based on the age, to determine the threshold.

For some applications, the threshold is selected from the group consisting of: at least 0.8 times an aerobic heart rate threshold, and at least 0.8 times an aerobic respiratory rate threshold.

For some applications, the threshold is selected from the group consisting of: at least an aerobic heart rate threshold and at least an aerobic respiratory rate threshold.

For some applications, the threshold is selected from the group consisting of: at least 0.7 times an anaerobic heart rate threshold, and at least 0.7 times an anaerobic respiratory rate threshold.

For some applications, the threshold is selected from the group consisting of: at least 0.8 times an anaerobic heart rate threshold, and at least 0.8 times an anaerobic respiratory rate threshold.

For some applications, the threshold is selected from the group consisting of: at least an anaerobic heart rate threshold, and at least an anaerobic respiratory rate threshold

For some applications:

the amount of time is a given percentage of time during a given time period, and

in response to the subject status indication, the computer processor is configured to vary an alert parameter selected from the group consisting of: the threshold, the given percentage of time, and the amount of time.

For some applications, the selected subject status indication is an indication that the subject has been continuously in a bed over a previous in-bed period of at least 15 hours.

For some applications:

the at least one selected subject status indication is the indication that the subject has been continuously in a bed over a previous in-bed period of at least 15 hours, and

in response to the selected subject status indication, the computer processor is configured to immediately generate the alert.

For some applications:

the selected subject status indication is an indication that the subject is not moving, and

in response to the indication that the subject is not moving, the computer processor is configured to generate the alert if the sensor signal indicates that the physiological parameter of the subject exceeds the threshold for at least 70% of the time during a time period of at least 15 minutes.

For some applications:

the selected subject status is an indication that the subject is awake and not moving, and

in response to the indication that the subject is awake and not moving, the computer processor is configured to generate the alert if the sensor signal indicates that the physiological parameter of the subject exceeds the threshold for at least 70% of the time during a time period of at least 60 minutes.

For some applications:

the selected subject status indication is an indication that the subject is not moving, and in response to the indication that the subject is not moving, the computer processor is configured to:
(a) determine the threshold to be a threshold selected from the group consisting of:
   (i) at least 0.7 times an anaerobic heart rate threshold, and
   (ii) at least 0.7 times an anaerobic respiratory rate threshold, and
(b) generate the alert if the sensor signal indicates that the physiological parameter of the subject exceeds the threshold for at least 70% of the time during a time period of at least 5 minutes.

For some applications:

the selected subject status is an indication that the subject is awake and not moving, and

in response to the indication that the subject is awake and not moving, the computer processor is configured to:
(a) determine the threshold to be a threshold selected from the group consisting of:
   (i) at least 0.7 times an anaerobic heart rate threshold, and
   (ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

For some applications:

the selected subject status indication is an indication that the subject is moving and asleep, and

in response to the indication that the subject is moving and asleep, the computer processor is configured to generate the alert if the sensor signal indicates that the physiological parameter of the subject exceeds the threshold for at least 70% of the time during a time period of at least 60 minutes.

For some applications:

the selected subject status indication is an indication that the subject is moving and asleep, and

in response to the indication that the subject is moving and asleep, the computer processor is configured to
(a) determine the threshold to be a threshold selected from the group consisting of:
   (i) at least 0.7 times an anaerobic heart rate threshold, and
   (ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

For some applications:

the selected subject status indication is an indication that the subject is awake and moving and in a bed, and

in response to the indication that the subject is awake and moving and in a bed, the computer processor is configured to generate the alert if the sensor signal indicates that the physiological parameter of the subject exceeds the threshold for at least 70% of the time during a time period of at least 60 minutes.

For some applications:

the selected subject status indication is an indication that the subject is awake and moving and in a bed, and

in response to the indication that the subject is awake and moving and in a bed, the computer processor is configured to
(a) determine the threshold to be a threshold selected from the group consisting of:
   (i) at least 0.7 times an anaerobic heart rate threshold, and
   (ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

For some applications:

the at least one selected subject status indication is an indication that the subject is asleep, not moving, and not in rapid eye movement (REM) sleep, and

in response to the indication that the subject is asleep, not moving, and not in REM sleep, the computer processor is configured to generate the alert immediately if the sensor signal indicates that the physiological parameter of the subject exceeds the threshold.

For some applications:

the at least one selected subject status indication is an indication that the subject is moving and asleep and not in rapid eye movement (REM) sleep, and

in response to the indication that the subject is moving and asleep and not in REM sleep, the computer processor is configured to generate the alert if the sensor signal indicates that the physiological parameter of the subject exceeds the threshold for at least 70% of the time during a time period of at least 5 minutes.

For some applications:

the at least one selected subject status indication is an indication that the subject is asleep, not moving, and in rapid eye movement (REM) sleep, and

in response to the indication that the subject is asleep, not moving and in REM sleep, the computer processor is configured to generate the alert if the sensor signal indicates that the physiological parameter of the subject exceeds the threshold for at least 70% of the time during a time period of at least 15 minutes.

For some applications:

the at least one selected subject status indication is an indication that the subject is moving and asleep, and in rapid eye movement (REM) sleep, and

in response to the indication that the subject is moving and asleep, and in REM sleep, the computer processor is configured to generate the alert if the sensor signal indicates that the physiological parameter of the subject exceeds the threshold for at least 70% of the time during a time period of at least 60 minutes.

For some applications:

the at least one selected subject status indication is an indication that the subject is asleep and in rapid eye movement (REM) sleep, and

in response to the indication that the subject is asleep and in REM sleep, the computer processor is configured to
(a) determine the threshold to be a threshold selected from the group consisting of:
   (i) at least 0.7 times an anaerobic heart rate threshold, and
   (ii) at least 0.7 times an anaerobic respiratory rate threshold, and
(b) generate the alert if the sensor signal indicates that the physiological parameter of the subject exceeds the threshold for at least 70% of the time during a time period of at least 5 minutes.

The present invention will be more fully understood from the following detailed description of applications thereof, taken together with the drawings, in which:

### BRIEF DESCRIPTION OF THE DRAWINGS

FIG. 1 is a schematic illustration of a system for monitoring a subject, in accordance with some applications of the present invention;

FIG. 2 is a graph depicting an exemplary model for reducing false positive and false negative alerts, in accordance with some applications of the present invention; and

FIG. 3 is a graph depicting a model for alert generation, in accordance with some applications of the present invention.

### DETAILED DESCRIPTION

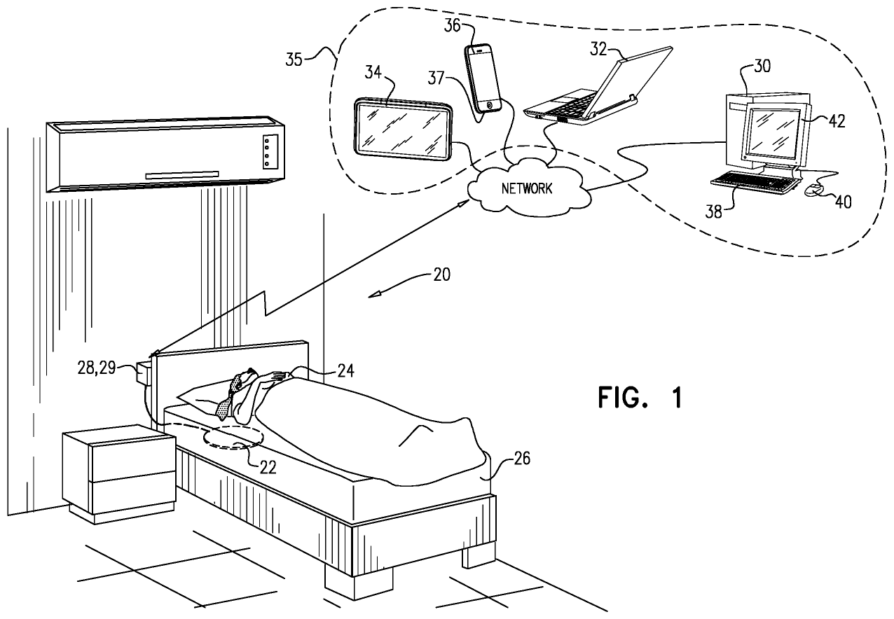

Reference is made to FIG. 1, which is a schematic illustration of subject-monitoring apparatus 20, in accordance with some applications of the present invention. Apparatus 20 is generally used to monitor a subject 24, while he or she is in a bed or lying on a resting surface. Apparatus 20 typically comprises a sensor 22 (e.g., a mechanical sensor, or an optical sensor) and a computer processor 28. For some applications, sensor 22 is disposed on or within the subject's bed, and configured to monitor the subject automatically, while he is in his bed. For example, sensor 22 may be a mechanical sensor, e.g., a motion sensor, disposed underneath the subject's mattress 26, such that the subject is monitored while he is lying upon the mattress, and while carrying out his normal sleeping routine, without the subject needing to perform an action to facilitate the monitoring that would not have otherwise been performed. For some applications, apparatus 20 is used to monitor subject 24, while he or she is in his or her bed in a home setting. For some applications, the subject-monitoring apparatus is used in a hospital setting.

Subject-monitoring apparatus 20 comprises a sensor 22 (e.g., a motion sensor) that is configured to monitor subject 24. Sensor 22 may be a motion sensor that is similar to sensors described in U.S. Pat. No. 8,882,684 to Halperin, which is incorporated herein by reference. The term “motion sensor” refers to a sensor that senses the subject's motion (e.g., motion due to the subject's cardiac cycle, respiratory cycle, or large-body motion of the subject), while the term “sensor” refers more generally to any type of sensor, e.g., a sensor that includes an electromyographic sensor and/or an imaging sensor.

Typically, sensor 22 includes a sensor that performs monitoring of the subject without contacting the subject or clothes the subject is wearing, and/or without viewing the subject or clothes the subject is wearing. For example, the sensor may perform the monitoring without having a direct line of sight of the subject's body, or the clothes that the subject is wearing. Further typically, the sensor performs monitoring of the subject without requiring subject compliance (i.e., without the subject needing to perform an action to facilitate the monitoring that would not have otherwise been performed). It is noted that, prior to the monitoring, certain actions (such as purchasing the sensor and placing the sensor under the subject's mattress) may need to be performed. The term “without requiring subject compliance” should not be interpreted as excluding such actions. Rather the term “without requiring subject compliance” should be interpreted as meaning that, once the sensor has been purchased, placed in a suitable position and activated, the sensor can be used to monitor the subject (e.g., to monitor the subject during repeated monitoring sessions), without the subject needing to perform any actions to facilitate the monitoring that would not have otherwise been performed.

For some applications, sensor 22 is disposed on or within the subject's bed, and configured to monitor the subject automatically, while she is in her bed. For example, sensor 22 may be disposed underneath the subject's mattress 26, such that the subject is monitored while she is lying upon the mattress, and while carrying out her normal sleeping routine, without the subject needing to perform an action to facilitate the monitoring that would not have otherwise been performed.

A computer processor 28, which acts as a control unit that performs the algorithms described herein, analyzes the signal from sensor 22. Typically, computer processor 28 communicates with a memory 29. For some applications, computer processor 28 is embodied in a desktop computer 30, a laptop computer 32, a tablet device 34, a smartphone 36, and/or a similar device that is programmed to perform the techniques described herein (e.g., by downloading a dedicated application or program to the device), such that the computer processor acts as a special-purpose computer processor. For some applications, as shown in FIG. 1, computer processor 28 is a dedicated computer processor that receives (and optionally analyzes) data from sensor 22, and communicates with computer processors of one or more of the aforementioned devices, which act as external devices.

For some applications, the subject (or another person, such as a care-giver) communicates with (e.g., sends data to and/or receives data from) computer processor 28 via a user interface device 35. As described, for some applications, computer processor 28 is embodied in a desktop computer 30, a laptop computer 32, a tablet device 34, a smartphone 36, and/or a similar device that is programmed to perform the techniques described herein. For such applications, components of the device (e.g., the touchscreen, the mouse, the keyboard, the speakers, the screen) typically act as user interface device 35. Alternatively, as shown in FIG. 1, computer processor 28 is a dedicated computer processor that receives (and optionally analyzes) data from sensor 22. For some such applications, the dedicated computer processor communicates with computer processors of one or more of the aforementioned external devices (e.g., via a network), and the user interfaces of the external devices (e.g., the touchscreen, the mouse, the keyboard, the speakers, the screen) are used by the subject, as user interface device 35, to communicate with the dedicated computer processor and vice versa. For some applications, in order to communicate with computer processor 28, the external devices are programmed to communicate with the dedicated computer processor (e.g., by downloading a dedicated application or program to the external device).

For some applications, user interface device 35 includes an input device such as a keyboard 38, a mouse 40, a joystick (not shown), a touchscreen device (such as smartphone 36 or tablet device 34), a touchpad (not shown), a trackball (not shown), a voice-command interface 37, and/or other types of user interfaces that are known in the art. For some applications, the user interface includes an output device such as a display (e.g., a monitor 42, a head-up display (not shown) and/or a head-mounted display (not shown)), and/or a different type of visual, text, graphics, tactile, audio, and/or video output device, e.g., speakers, headphones, smartphone 36, or tablet device 34. For some applications, the user interface acts as both an input device and an output device. For some applications, the processor generates an output on a computer-readable medium (e.g., a non-transitory computer-readable medium), such as a disk, or a portable USB drive.

For some applications, sensor 22 continuously monitors subject 24 and generates a sensor signal in response to the monitoring. (The use of the word “continuously” in the present application and in the claims does not exclude occasional interruptions, e.g., due to loss of signal, mechanical interruptions, or brief diversions of apparatus 20 to perform other tasks.) Computer processor 28 receives the sensor signal, and derives from the sensor signal data related to the subject's heart rate and/or respiratory rate. Computer processor 28 determines a threshold, i.e., sets a specific threshold, that is:
(a) at least 0.7 times an aerobic heart rate threshold, e.g., at least 0.8 times an aerobic heart rate threshold, e.g., at least an aerobic heart rate threshold,
(b) at least 0.7 times an anaerobic heart rate threshold, e.g., at least 0.8 times an anaerobic heart rate threshold, e.g., at least an anaerobic heart rate threshold,
(c) at least 0.7 times an aerobic respiratory rate threshold, e.g., at least 0.8 times an aerobic respiratory rate threshold, e.g., at least an aerobic respiratory rate threshold, and/or
(d) at least 0.7 times an anaerobic respiratory rate threshold, e.g., at least 0.8 times an anaerobic respiratory rate threshold, e.g., at least an anaerobic respiratory rate threshold.

As used in the present application, including in the claims, at least 0.7 times an aerobic and/or anaerobic heart/respiratory rate threshold is given to mean at least 0.7 of a known or calculated threshold, e.g., a known or calculated aerobic and/or anaerobic threshold for a given subject. Thus, within the scope of the present invention is setting a threshold that is above 0.7 times the known or calculated threshold, e.g., 0.8 times the known or calculated threshold, e.g., 0.95 times the known or calculated threshold, e.g., the known or calculated threshold itself, or higher.

Typically, computer processor 28 determines the respective thresholds for a given subject 24 based on aerobic and anaerobic heart/respiratory zones of the given subject 24. The aerobic and anaerobic heart/respiratory zones of the given subject 24 may be determined, for example, by using a look-up table, or calculated using the following exemplary series of equations:

HR(max)=(220−Age)

RR(max)=(220−Age)/3

HRR=HR(max)−HR(min)

RRR=RR(max)−RR(min)

HRZ(aerobic)=[HR(min)+(0.7*HRR),HR(min)+(0.8*HRR)]

HRZ(anaerobic)=[(HR(min)+(0.8*HRR)),(HR(min)+(0.9*HRR))]

RRZ(aerobic)=[RR(min)+(0.7*HRR),RR(min)+(0.8*HRR)]

RRZ(anaerobic)=[(RR(min)+(0.8*HRR)),(RR(min)+(0.9*HRR))]

where:
HR(max) is the maximal heart rate of given subject 24,
RR(max) is the maximal respiratory rate of given subject 24,
“Age” refers to the age of given subject 24,
HR(min) is the minimum heart rate of given subject 24 which, for some applications, is determined to be the heart rate while given subject 24 is at rest and not moving during the second half of the night while in deep sleep,
RR(min) is the minimum respiratory rate of given subject 24 which, for some applications, is determined to be the respiratory rate while given subject 24 is at rest and not moving during the second half of the night while in deep sleep,
HRR is the heart rate range of given subject 24,
RRR is the respiratory rate range of given subject 24,
HRZ(aerobic) is the aerobic heart rate zone of given subject 24,
HRZ(anaerobic) is the anaerobic heart rate zone of given subject 24,
RRZ(aerobic) is the aerobic respiratory rate zone of given subject 24,
RRZ(anaerobic) is the anaerobic respiratory rate zone of given subject 24, and
The terminology [x, y] means the range from x to y.

For some applications, alternatively or additionally to using age of subject 24 as the input for calculating the respective thresholds, other parameters such as weight of subject 24, may be used. Alternatively or additionally, fixed thresholds may be used that are not subject-specific. Alternatively or additionally, parameters such as subject 24 being considered a cardiac-risk patient, or the results of a medical test, e.g., blood test, or cardiac stress test, may be used as input for calculating the respective ranges and thresholds.

The inventors have realized that an aerobic heart rate and/or an aerobic respiratory rate (a) is not expected for more than one to two hours while subject 24 is in bed, (b) is not expected for more than 30 minutes while subject 24 is at rest without movement, and (c) is not expected at all while subject 24 is undergoing non-REM sleep. The inventors have also realized that an anaerobic heart rate and/or an anaerobic respiratory rate (a) is not expected for more than 30 minutes while subject 24 is in bed, (b) is not expected for more than 5 minutes while subject 24 is at rest and without movement, and (c) is not expected at all while subject 24 is undergoing non-REM sleep.

Thus, sensor 22, being typically disposed on or under subject's 24 bed or other resting surface, e.g., on or under mattress 26, continuously monitors subject 24 while subject 24 is in their bed or other resting surface. Computer processor 28 typically generates an alert, e.g., a visual alert, an audio alert, or a vibrational alert, if the sensor signal indicates that the heart rate and/or respiratory rate of subject 24 exceeds a respective threshold for a given percentage (e.g., 70%, e.g., 80%) of the time during a time period of at least 15 minutes, e.g., at least 30 minutes, e.g., at least 60 minutes.

As described hereinabove, using additional parameters such as for example, sleep stage of a sleeping subject and motion information allows providing a timely, accurate alert with few false alarms. For some applications, computer processor 28 derives from the sensor signal at least one subject status indication selected from the following list:
an indication that the subject is moving and asleep,
an indication that the subject is not moving,
an indication that the subject is awake and not moving,
an indication that the subject is asleep,
an indication that the subject is awake and moving and in a bed,
an indication of a particular sleep stage of the subject while the subject is asleep, and
an indication that the subject has been continuously in a bed over a previous in-bed period of at least 15 hours.

Computer processor 28 typically generates the alert if the sensor signal indicates that the heart rate and/or respiratory rate of subject 24 exceeds the determined threshold for an amount of time determined in response to the subject status indication.

For some applications, the amount of time is a given percentage of time during a given time period, and in response to the subject status indication, computer processor 28 varies an alert parameter, such as the threshold, the given percentage of time, and/or the amount of time.

The following bulleted list contains examples of how computer processor 28 may utilize the subject status indications to generate the alert. For all of the examples in the following list, the threshold is determined as:

(a) at least 0.7 times an aerobic heart rate threshold, e.g., at least 0.8 times an aerobic heart rate threshold, e.g., at least an aerobic heart rate threshold,

(b) at least 0.7 times an anaerobic heart rate threshold, e.g., at least 0.8 times an anaerobic heart rate threshold, e.g., at least an anaerobic heart rate threshold,

(c) at least 0.7 times an aerobic respiratory rate threshold, e.g., at least 0.8 times an aerobic respiratory rate threshold, e.g., at least an aerobic respiratory rate threshold, and/or

(d) at least 0.7 times an anaerobic respiratory rate threshold, e.g., at least 0.8 times an anaerobic respiratory rate threshold, e.g., at least an anaerobic respiratory rate threshold.
If the selected subject status indication is an indication that the subject is not moving, computer processor 28 generates the alert if the sensor signal indicates that the heart rate and/or respiratory rate of subject 24 exceeds the threshold for at least 70% of the time during a time period of at least 15 minutes, e.g., at least 30 minutes.
If the selected subject status indication is an indication that the subject is not moving, computer processor 28 may use a relatively higher threshold, e.g., 0.7 times an anaerobic heart rate threshold and/or at least 0.7 times an anaerobic respiratory rate threshold, and may generate the alert if the sensor signal indicates that the heart rate and/or respiratory rate of subject 24 exceeds the threshold for at least 70% of the time during a time period of at least 5 minutes, e.g., even if the time period is still quite short, e.g., less than 30 minutes or less than 15 minutes.
If the selected subject status indication is an indication that the subject is awake and not moving, computer processor 28 generates the alert if the sensor signal indicates that the heart rate and/or respiratory rate of subject 24 exceeds the threshold for at least 70% of the time during a time period of at least 60 minutes, e.g., at least 3 hours.
If the selected subject status indication is an indication that the subject is awake and not moving, computer processor 28 may use a relatively higher threshold, e.g., 0.7 times an anaerobic heart rate threshold and/or at least 0.7 times an anaerobic respiratory rate threshold, and may generate the alert if the sensor signal indicates that the heart rate and/or respiratory rate of subject 24 exceeds the threshold for at least 70% of the time during a time period of at least 15 minutes, e.g., at least 30 minutes, e.g., even if the time period is still relatively short, e.g., less than 60 minutes.
If the selected subject status indication is an indication that the subject is moving and asleep, computer processor 28 generates the alert if the sensor signal indicates that the heart rate and/or respiratory rate of subject 24 exceeds the threshold for at least 70% of the time during a time period of at least 60 minutes, e.g., at least 2 hours, e.g., at least 3 hours.
If the selected subject status indication is an indication that the subject is moving and asleep, computer processor 28 may use a relatively higher threshold, e.g., 0.7 times an anaerobic heart rate threshold and/or at least 0.7 times an anaerobic respiratory rate threshold, and may generate the alert if the sensor signal indicates that the heart rate and/or respiratory rate of subject 24 exceeds the threshold for at least 70% of the time during a time period of at least 15 minutes, e.g., at least 30 minutes, e.g., even if the time period is still relatively short, e.g., less than 60 minutes.
If the selected subject status indication is an indication that the subject is awake and moving and in a bed, computer processor 28 generates the alert if the sensor signal indicates that the heart rate and/or respiratory rate of subject 24 exceeds the threshold for at least 70% of the time during a time period of at least 60 minutes, e.g., at least 3 hours, e.g., at least 6 hours.
If the selected subject status indication is an indication that the subject is awake and moving and in a bed, computer processor 28 may use a relatively higher threshold, e.g., 0.7 times an anaerobic heart rate threshold and/or at least 0.7 times an anaerobic respiratory rate threshold, and may generate the alert if the sensor signal indicates that the heart rate and/or respiratory rate of subject 24 exceeds the threshold for at least 70% of the time during a time period of at least 15 minutes, e.g., at least 30 minutes, e.g., even if the time period is still relatively short, e.g., less than 60 minutes.
If the subject status indication is an indication that the subject is asleep, not moving, and not in rapid eye movement (REM) sleep, computer processor 28 may generate the alert immediately if the sensor signal indicates that the physiological parameter of the subject exceeds the threshold.
If the selected subject status indication is an indication that the subject is moving and asleep and not in rapid eye movement (REM) sleep, computer processor 28 may generate the alert if the sensor signal indicates that the heart rate and/or respiratory rate of subject 24 exceeds the threshold for at least 70% of the time during a time period of at least 5 minutes, e.g., even if the time period is still quite short, e.g., less than 30 minutes or less than 15 minutes.
If the selected subject status indication is an indication that the subject is asleep, not moving, and in rapid eye movement (REM) sleep, computer processor 28 may generate the alert if the sensor signal indicates that the heart rate and/or respiratory rate of subject 24 exceeds the threshold for at least 70% of the time during a time period of at least 15 minutes, e.g., at least 30 minutes, e.g., even if the time period is still relatively short, e.g., less than 60 minutes.
If the selected subject status indication is an indication that the subject is moving and asleep, and in rapid eye movement (REM) sleep, computer processor 28 may generate the alert if the sensor signal indicates that the heart rate and/or respiratory rate of subject 24 exceeds the threshold for at least 70% of the time during a time period of at least 30 minutes, e.g., at least 60 minutes.
If the selected subject status indication is an indication that the subject is asleep and in rapid eye movement (REM) sleep, computer processor 28 may use a relatively higher threshold, e.g., 0.7 times an anaerobic heart rate threshold and/or at least 0.7 times an anaerobic respiratory rate threshold, and may generate the alert if the sensor signal indicates that the heart rate and/or respiratory rate of subject 24 exceeds the threshold for at least 70% of the time during a time period of at least 5 minutes, e.g., even if the time period is still quite short, e.g., less than 30 minutes or less than 15 minutes.
If the subject status indication is an indication that the subject has been continuously in a bed over a previous in-bed period of at least 15 hours (e.g., at least 16 hours, e.g., at least 18 hours, e.g., at least 24 hours), computer processor 28 generates the alert immediately, e.g., regardless of the heart rate and/or respiratory rate of subject 24.
If the subject status indication is an indication that the subject has been continuously in a bed over a previous in-bed period of at least 15 hours, computer processor 28 may generate the alert if the sensor signal indicates that the heart rate and/or respiratory rate of subject 24 exceeds the threshold for at least 70% of the time during a time period of at least 60 minutes, e.g., at least 2 hours.

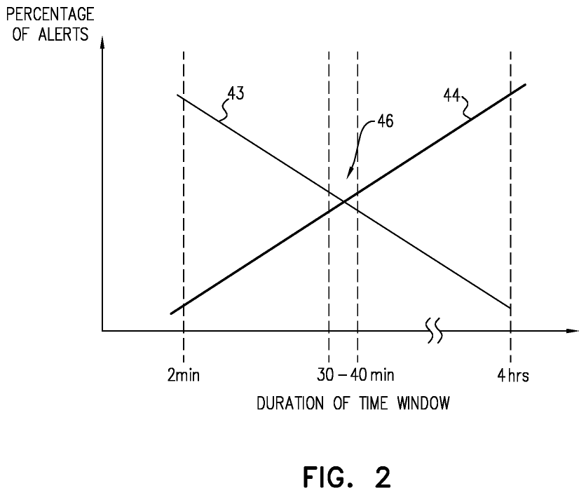

Reference is now made to FIG. 2, which is a graph showing a theoretical example of how the time windows are set so as to reduce the number of false positive alerts and false negative alerts. Curve 43 on the graph shows for any given time window (plotted along the x-axis) what percentage of alerts are false positive alerts. As can be seen, if the time window is relatively small, e.g., 2 minutes, computer processor 28 may generate an alert while the subject, for example, may have briefly experienced an elevated heart and/or respiratory rate for a reason unconnected to an adverse clinical condition. The lowest percentage of false positive alerts would be if the time window were set relatively large, e.g., 4 hours. Curve 44 on the graph shows for any given time window what percentage of alerts are false negatives. The lowest number of false negative alerts would occur if the time window were set very small, e.g., almost every case of an elevated heart rate being indicative of an onset or worsening adverse clinical condition would trigger an alert, however as described above, setting such a small time window increases the changes of false positive alerts. If a relatively large time window is used, e.g., 4 hours, the percentage of false negative alerts would be high, i.e., many cases where it would have otherwise been advantageous to the health of the patient to alert, may be missed. Thus, the inventors have realized that, for some applications, a time window that is at least 30 minutes is useful to reduce false positives without producing an unacceptable excess of false negatives. Similarly, the inventors have realized that, for some applications, a time window that is less than 40 minutes is useful to reduce false negatives without producing an unacceptable excess of false positives. Thus, for some applications of the present invention, a range of about 30-40 minutes, marked by arrow 46, provides a balance between reducing both false positive alerts and false negative alerts. For this exemplary model, for both curves the alerts are generated when the subject's heart/respiratory rate exceeds the threshold during 70% of the time window.

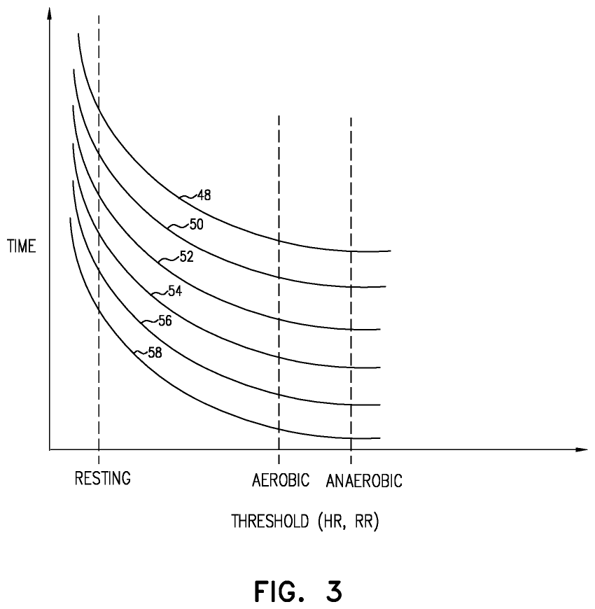

Reference is now made to FIG. 3, which is a graph showing an exemplary model of curves, which plots the threshold against the amount of time the subject's heart/respiratory rate exceeds the threshold before an alert is generated. Each curve represents a different subject status indication. Curve 48 is for a subject 24 who is awake and moving. Curve 50 is for a subject 24 who is asleep, moving, and in REM sleep. Curve 52 is for a subject 24 who is awake and not moving. Curve 54 is for a subject 24 who is asleep, not moving, and in REM sleep. Curve 56 is for a subject 24 who is asleep, moving and not in REM sleep. Curve 58 is for a subject 24 who is asleep, not moving, and not in REM sleep. As is shown by the progression of the curves, generally the more awake and active the subject is the longer the system waits before generating an alert. This is in line with the inventors' hypothesis that elevated heart/respiratory rates near or exceeding the aerobic and anaerobic zones while the subject is at rest and not active may be indicative of an onset or worsening of an adverse clinical condition, as described hereinabove.

Also shown on the graph of FIG. 3 is a resting threshold. For some applications computer processor 28 determines based on the sensor signal, for any given subject 24, a resting threshold that is within a resting zone, e.g., a resting heart rate threshold that is within a resting heart rate zone of given subject 24, and/or a resting respiratory rate threshold that is within a resting respiratory rate zone of given subject 24. Computer processor 28 generates an alert if the sensor signal indicates that the heart rate and/or respiratory rate of subject 24 exceeds a respective resting threshold for at least 70% of the time during a time period of at least 3 hours, e.g., at least 6 hours.

The inventors have realized that a person's heart/respiratory rate should be in the resting zone for at least 50% percent of the time over a time period of 3-6 hours in bed, and should always be in the resting zone while the subject is undergoing non-REM sleep. However, even within the resting zone, if a subject's heart rate is slightly elevated, yet still within the resting zone, for a substantial amount of time, it may be indicative of the onset or worsening of an adverse clinical condition. For example, computer processor 28 may generate an alert if a given subject's heart/respiratory rate is within the resting zone but above the resting threshold, and does not decrease back down to below the resting threshold for more than three hours, or if the subject's heart rate is in the resting zone but above the resting threshold while in non-REM sleep. (The scope of the present invention does not exclude apparatus or a method that additionally alerts if a subject's heart/respiratory rate is above the resting threshold and outside the resting zone for at least 70% of the time period.)

Typically, the resting threshold for a give subject is calculated based on the subject's age, maximal heart/respiratory rate and minimum heart/respiratory rate. Typically, the lower end of the resting heart rate zone is a resting heart rate that is somewhere between 45 beats per minute and 80 beats per minute. The higher end of the resting zone is typically above the lower end of the resting heart rate zone by up to 50% of the difference between the maximal heart rate and the resting heart rate of a given subject 24. As described hereinabove, for some applications, the minimum heart rate of a given subject 24 is determined to be the heart rate while given subject 24 is at rest and not moving during the second half of the night while in deep sleep, and the minimum respiratory rate of given subject 24 is determined to be the respiratory rate while given subject 24 is at rest and not moving during the second half of the night while in deep sleep.

Alternatively or additionally, the resting heart/respiratory rate threshold may be determined as the lowest respective heart/respiratory rate of the subject in a previous given time period, e.g., a previous 5-8 hours, e.g., during the second half of the night, e.g., between a first time of day that is between 9:00 pm and 1:00 am, and a second time of day that is between 5:00 and 9:00 am, e.g., between 11:00 pm and 6:00 am.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., computer processor 28) coupled directly or indirectly to memory elements of memory 29 through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the methods described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the methods described in the present application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the methods described in the present application.

Computer processor 28 is typically a hardware device programmed with computer program instructions to produce a special purpose computer. For example, when programmed to perform the methods described herein, the computer processor typically acts as a special purpose computer processor. Typically, the operations described herein that are performed by computer processors transform the physical state of memory 29, which is a real physical article, to have a different magnetic polarity, electrical charge, or the like depending on the technology of the memory that is used.

It will be appreciated by subjects skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to subjects skilled in the art upon reading the foregoing description.

1. Apparatus for monitoring a subject, the apparatus comprising:
(A) a sensor, configured to continuously monitor the subject, and to generate a sensor signal in response to the monitoring; and
(B) a computer processor, configured:
to receive the sensor signal,
to derive from the sensor signal data related to one or more physiological parameters of the subject selected from the group consisting of: a heart rate of the subject, and a respiration rate of the subject,
to derive from the sensor signal at least one subject status indication selected from the group consisting of: an indication that the subject is moving and asleep, an indication that the subject is not moving, an indication that the subject is awake and not moving, an indication that the subject is asleep, an indication that the subject is awake and moving and in a bed, an indication of a particular sleep stage of the subject while the subject is asleep, and an indication that the subject has been continuously in a bed over a previous in-bed period of at least 15 hours,
to determine a threshold selected from the group consisting of: at least 0.7 times an aerobic heart rate threshold, and at least 0.7 times an aerobic respiratory rate threshold, and
to generate an alert if the sensor signal indicates that the physiological parameter of the subject exceeds the threshold for an amount of time determined in response to the subject status indication.

2. The apparatus according to claim 1, wherein the sensor is configured to be disposed at a location selected from the group consisting of: upon the subject's bed, and within the subject's bed.

3. The apparatus according to claim 1, wherein the computer processor is configured to receive an age of the subject and, based on the age, to determine the threshold.

4. The apparatus according to claim 1, wherein the threshold is selected from the group consisting of: at least 0.8 times an aerobic heart rate threshold, and at least 0.8 times an aerobic respiratory rate threshold.

5. (canceled)

6. The apparatus according to claim 1, wherein the threshold is selected from the group consisting of: at least 0.7 times an anaerobic heart rate threshold, and at least 0.7 times an anaerobic respiratory rate threshold.

7-8. (canceled)

9. The apparatus according to claim 1, wherein:
the amount of time is a given percentage of time during a given time period, and
in response to the subject status indication, the computer processor is configured to vary an alert parameter selected from the group consisting of: the threshold, the given percentage of time, and the amount of time.

10-11. (canceled)

12. The apparatus according to claim 9, wherein:
the selected subject status indication is an indication that the subject is not moving, and
in response to the indication that the subject is not moving, the computer processor is configured to generate the alert if the sensor signal indicates that the physiological parameter of the subject exceeds the threshold for at least 70% of the time during a time period of at least 15 minutes.

13. (canceled)

14. The apparatus according to claim 9, wherein:
the selected subject status indication is an indication that the subject is not moving, and
in response to the indication that the subject is not moving, the computer processor is configured to:
(a) determine the threshold to be a threshold selected from the group consisting of:
(i) at least 0.7 times an anaerobic heart rate threshold, and
(ii) at least 0.7 times an anaerobic respiratory rate threshold, and
(b) generate the alert if the sensor signal indicates that the physiological parameter of the subject exceeds the threshold for at least 70% of the time during a time period of at least 5 minutes.

15. The apparatus according to claim 9, wherein:
the selected subject status is an indication that the subject is awake and not moving, and
in response to the indication that the subject is awake and not moving, the computer processor is configured to:
(a) determine the threshold to be a threshold selected from the group consisting of:
(i) at least 0.7 times an anaerobic heart rate threshold, and
(ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

16. (canceled)

17. The apparatus according to claim 9, wherein:
the selected subject status indication is an indication that the subject is moving and asleep, and
in response to the indication that the subject is moving and asleep, the computer processor is configured to
(a) determine the threshold to be a threshold selected from the group consisting of:
(i) at least 0.7 times an anaerobic heart rate threshold, and
(ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

18. The apparatus according to claim 9, wherein:
the selected subject status indication is an indication that the subject is awake and moving and in a bed, and
in response to the indication that the subject is awake and moving and in a bed, the computer processor is configured to generate the alert if the sensor signal indicates that the physiological parameter of the subject exceeds the threshold for at least 70% of the time during a time period of at least 60 minutes.

19. The apparatus according to claim 9, wherein:
the selected subject status indication is an indication that the subject is awake and moving and in a bed, and
in response to the indication that the subject is awake and moving and in a bed, the computer processor is configured to
(a) determine the threshold to be a threshold selected from the group consisting of:
(i) at least 0.7 times an anaerobic heart rate threshold, and
(ii) at least 0.7 times an anaerobic respiratory rate threshold, and
(b) generate the alert if the sensor signal indicates that the physiological parameter of the subject exceeds the threshold for at least 70% of the time during a time period of at least 15 minutes.

20. The apparatus according to claim 9, wherein:
the at least one selected subject status indication is an indication that the subject is asleep, not moving, and not in rapid eye movement (REM) sleep, and
in response to the indication that the subject is asleep, not moving, and not in REM sleep, the computer processor is configured to generate the alert immediately if the sensor signal indicates that the physiological parameter of the subject exceeds the threshold.

21. The apparatus according to claim 9, wherein:
the at least one selected subject status indication is an indication that the subject is moving and asleep and not in rapid eye movement (REM) sleep, and
in response to the indication that the subject is moving and asleep and not in REM sleep, the computer processor is configured to generate the alert if the sensor signal indicates that the physiological parameter of the subject exceeds the threshold for at least 70% of the time during a time period of at least 5 minutes.

22. The apparatus according to claim 9, wherein:
the at least one selected subject status indication is an indication that the subject is asleep, not moving, and in rapid eye movement (REM) sleep, and
in response to the indication that the subject is asleep, not moving and in REM sleep, the computer processor is configured to generate the alert if the sensor signal indicates that the physiological parameter of the subject exceeds the threshold for at least 70% of the time during a time period of at least 15 minutes.

23-24. (canceled)

25. Apparatus for monitoring a subject, the apparatus comprising:
(A) a sensor configured to continuously monitor the subject, and to generate a sensor signal in response to the monitoring; and
(B) a computer processor, configured:
to receive the sensor signal,
to derive from the sensor signal data related to one or more physiological parameters of the subject selected from the group consisting of: a heart rate of the subject, and a respiration rate of the subject,
to determine a threshold selected from the group consisting of: at least 0.7 times an aerobic heart rate threshold, and at least 0.7 times an aerobic respiratory rate threshold, and
to generate an alert if the sensor signal indicates that the physiological parameter of the subject exceeds the threshold for a given percentage of time during a time period when the subject is not moving for at least 15 minutes.

26. The apparatus according to claim 25, wherein the sensor is configured to be disposed at a location selected from the group consisting of: upon a bed of the subject, and within the subject's bed.

27. The apparatus according to claim 25, wherein the sensor is configured to continuously monitor the subject, without contacting or viewing the subject, and to generate the sensor signal in response to the monitoring.

28. The apparatus according to claim 25, wherein the computer processor is configured to receive an age of the subject and, based on the age, to determine the threshold.

29. The apparatus according to claim 25, wherein the time period is at least 30 minutes.

30-31. (canceled)

32. The apparatus according to claim 25, wherein the threshold is selected from the group consisting of: at least 0.7 times an anaerobic heart rate threshold, and at least 0.7 times an anaerobic respiratory rate threshold.

33-47. (canceled)
